(19) United States
(12) Patent Application Publication
(10) Pub. No.: US 2020/0176128 A1
(43) Pub. Date: Jun. 4, 2020

(54) Identifying Drug Side Effects

(71) Applicant: Ravipal Soin, Redmond, WA (US)

(72) Inventor: Ravipal Soin, Redmond, WA (US)

(21) Appl. No.: 16/730,657

(22) Filed: Dec. 30, 2019

Related U.S. Application Data
(60) Provisional application No. 62/774,281, filed on Dec. 2, 2018.

(51) Int. Cl. G16H 70/40; G06F 16/953; G06F 16/951; G16H 10/60; G06F 40/30; G06Q 50/00

(52) U.S. Cl. CPC G16H 70/40; G06F 16/953; G06Q 50/01; G16H 10/60; G06F 40/30; G06F 16/951

(57) ABSTRACT

Side effects of pharmaceuticals may be investigated or discovered by analysis of internet discussions between patients.

### FIELD

The disclosure relates to identifying side effects for a drug.

### BACKGROUND

In 2014, there were nearly 4.8 million drug-related Emergency Department (ED) visits in the US. These visits included reports of drug abuse, adverse reactions to drugs, or other drug-related consequences. Almost 50 percent were attributed to adverse reactions to pharmaceuticals taken as prescribed, and 45 percent involved drug abuse. Drug Abuse Warning Network (DAWN) estimates that of the 2.2 million drug abuse visits in 2014, 27.1 percent involved nonmedical use of pharmaceuticals (i.e., prescription or OTC medications, dietary supplements). ED visits involving nonmedical use of pharmaceuticals (either alone or in combination with another drug) increased 98.4 percent between 2009 and 2014, from 627,291 visits to over 1.4 million, respectively. ED visits involving adverse reactions to pharmaceuticals increased 82.9 percent between 2005 and 2009, from 1,250,377 to 2,287,273 visits, respectively. The majority of drug-related ED visits were made by patients 21 or older (80.9 percent, or 3,717,030 visits). Patients aged 20 or younger accounted for 19.1 percent (877,802 visits) of all drug-related visits in 2014. ED visits involving adverse reactions to pharmaceuticals increased 84.9 percent between 2009 and 2014, from 1.2 million visits to over 2.3 million visits. The majority of adverse reaction visits were made by patients 21 or older, particularly among patients 65 or older; the rate increased 89.7 percent from 2009 to 2014 among this age group.

### SUMMARY

There are over 2.3 billion drugs prescribed by US physicians annually, with 2.4 billion posts by patients discussing their experience with drugs in online community forums. Just as disease outbreaks and vaccinations have been successfully modeled based on Google searches, these online discussions form a valuable source for mining patient knowledge about potential drug side effects, not on the drug label.

In one aspect, determining whether a search drug has a side effect may include searching a target website to identify pages matching the search drug, searching the identified pages for text matching the side effect, and determining relevance of the side effect by comparing the fraction of identified pages that match the side effect to a threshold, wherein a fraction of identified pages greater than or about equal to the threshold indicates that the side effect is relevant to the search drug. The determination may further include accessing a database of drugs or of side effects to obtain the drug or side effect to be searched. The search drug may be, for example, an active ingredient or an inactive ingredient. The target website may include health-related user-generated content, such as a health-related forum or a social community. Identifying pages matching the search drug may include identifying a drug name field in a structured page on the target website or matching the name of the drug to text on the website. Searching the identified pages for text matching the side effect may include preprocessing the identified pages to normalize text, for example, by a Porter stemmer algorithm. Searching the identified pages for text matching the side effect may include identifying text strings having elements that overlap elements of the side effect, or may include using semantic analysis to determine whether the text indicates that the side effect did not occur, in which case the determination may be that the text does not match the side effect. The threshold may be determined using the Rocchio method. The method may further include searching the target website to identify pages matching a second drug, or pages matching both drugs.

In another aspect, a system for determining whether a search drug may have a side effect may include a first search engine that searches a target website to identify pages matching the search drug, a second search engine that searches the identified pages for text matching the side effect, and a relevance calculator that determines relevance of the search side effect by comparing the fraction of identified pages that match the side effect to a threshold. A fraction of identified pages greater than or about equal to the threshold may indicate that the side effect is relevant to the search drug.

In another aspect, a method for constructing a side effect database for a group of drugs may include obtaining a side effect lexicon including a listing of possible side effects, creating a drug database including a record for each drug of the group of drugs, and for each drug of the group of drugs, identifying a plurality of web pages that include a discussion of the drug, and for each pair of web page and side effect, locating any text strings in the web page that match the side effect calculating a relevance of each side effect to the drug by considering located matches for all web pages that include a discussion of the drug, and if the calculated relevance exceeds a threshold, storing an indicator of the calculated relevance of the side effect to the drug in the database.

### BRIEF DESCRIPTION OF THE DRAWING

FIG. 1 is a block diagram of one embodiment of a system.

FIG. 2 is a block diagram of one embodiment of a method of creating a Knowledge Base.

FIG. 3 is a block diagram of one embodiment of a method of identifying discussions of drug side effects.

FIG. 4 is a graph showing mentions of heart rhythm symptoms online related to Darvocet.

FIG. 5 shows a small portion of a table containing drug-disease interaction data.

### DETAILED DESCRIPTION

A more particular description of certain embodiments of Identifying Drug Side Effects may be had by reference to the embodiments described below, and those shown in the drawings that form a part of this specification, in which like numerals represent like objects. It is understood that the description and drawings represent example implementations and are not to be understood as limiting. Drawings are not drawn to scale unless otherwise noted herein.

Notifying patients and physicians of potential drug effects is an important step in improving healthcare quality and delivery. While drugs can treat human diseases through chemical interactions between the ingredients and intended targets in the human body, the ingredients could unexpectedly interact with off-targets, which may cause adverse drug side effects. Patients may discuss possible drug side effects in health forums, on social media pages, or elsewhere on the internet. These discussions represent a previously largely untapped source of drug side effect data.

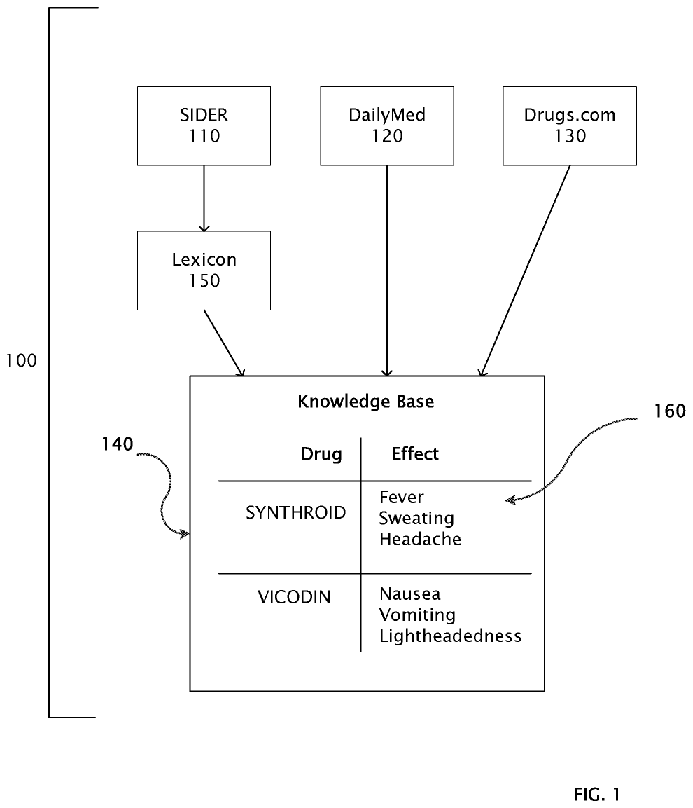

One embodiment of System 100 is shown in FIG. 1. In order to collect drug side effect data from patient experiences, Knowledge Base 140, that includes most of the known drug side effects, may be built. Online sources may provide information related to drug side effects. For example, the “Side Effect Resource” found at sideeffects.embl.de, SIDER 110, may contain extracted drug side effects from public documents and may provide the information in a well-structured format. DailyMed 120, which may be found at dailymed.nlm.nih.gov, may provide high-quality information about drugs approved by the Food and Drug Administration (FDA), including FDA labels. Drugs.com 130 is a popular drug-related website.

In reviewing the information from these three sources, it may be found that none of them contain all the drug-related information. Moreover, the language used to describe side effects may be different in different sources. For example, the terms used in DailyMed, which come from FDA drug labels, are often more formal, while the terms used in Drugs.com are more conversational since they come from the patients. Thus, it may be helpful to integrate the information from all these sources to construct a more complete Knowledge Base 140.

Among these three sources, only SIDER 110 provides structured information that makes it possible to extract drug names and side effects directly. Unfortunately, the other two sources are unstructured, so it is more challenging to extract drug names and side effects from them. However, most pages from DailyMed 120 and Drugs.com 130 are organized based on single drugs. Each page discusses the information of a single drug, and drug names are often mentioned in specific fields such as “title,” “drug,” or “drug name” in the HTML pages. Thus, a simple yet effective drug name extraction strategy may be to utilize the HTML template of each web source, identify the field related to drug names, and use these field values as drug names.

Unlike drug names that are often the values of specific fields, side effect names may be scattered in the plain text with noisy terms such as drug descriptions or drug labels. Thus, the drug name extraction method described above would not work well for side effect name extraction. To solve the problem, we use a Lexicon 150 to extract drug side effect names from the plain text. In the implementation described below, the side effect names from SIDER 110 may be used as Lexicon 150. SIDER 110 may be one of the most representative resources about drug side effects, and it may contain about 1,450 side effect names, which may be labeled as such. Additional side effects may be added to the Lexicon 150. Although the method described below uses the SIDER database, other databases of drug effects may also be used.

Lexicon 150 may be used to match the pages from those online sources, for example, SIDER 110, DailyMed 120, and Drugs.com 130, and decide whether a page matches a particular drug side effect. In some embodiments, instead of using only exact matching for side effect names, pre-processing the documents using a method such as Porter stemmer may be used, which may normalize the terms and make it possible to match terms with the same stem form, for example, “fevers” and “fever.” Moreover, instead of using exact string matching, in some embodiments, similarities between strings based on their overlapped terms may be computed. This strategy may allow identification of variants of a side effect such as “lung cancer” and “cancer of lung.” After extracting drugs and side effects, an integrated Knowledge Base 140 of drug side effects with a list 160 of drugs and their associated side effects may be constructed.

Health-related user-generated content, such as that found in thousands of openly available health forums and blogs, may be crawled to search for side effect data. Discussion forums may yield the richest source of side effect discussions, but social media such as Facebook, Twitter, Tumblr, and Reddit may also yield side effect data. Intuitively, if a particular side effect is indeed associated with the drug, more people will mention it in the online discussions. Thus, relevant side effects should have higher discussion frequency than non-relevant side effects.

Commonly used classification methods may include discriminative methods with the goal of directly modeling the boundary between the two categories. In some embodiments, the Rocchio method may be used, which may decide the label of a new data point based on the distance of the data point to the centroid of each category. Specifically, given a drug, a training dataset may be constructed, based on the information about the drug from Knowledge Base 140. For each of the drug's known side effects, i.e., effects appearing in list 160, online discussions may be collected, and then their average discussion frequency—the average fraction of discussions that mention the side effect under consideration—may be computed). The same procedure for the unknown side effects of the drug, i.e., side effects that appear in lexicon 150 but are not included in list 160, may be computed.

Once the side effect frequencies have been calculated, whether a side effect is relevant to the drug may be determined. A discussion frequency may be compared with the average frequency of known side effects and that of unknown side effects. If it is closer to the average discussion frequency of the known side effects, this side effect will be classified as relevant. Otherwise, the side effect will be classified as non-relevant. Any side effect classified as relevant that does not appear in the list of side effects in Knowledge Base 140 is potentially a heretofore unrecognized side effect.

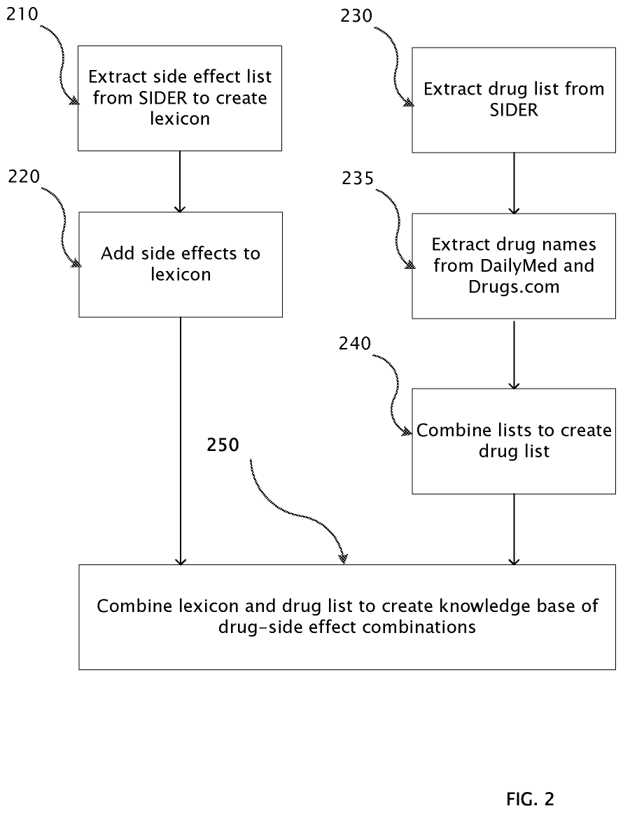

FIG. 2 is a block diagram of the method of constructing the Knowledge Base described in connection with the system of FIG. 1. As described above, a lexicon 150 may be created 210 of the list of side effects in SIDER 110, and additional side effects from any other sources may be added 220. The drug lists from the structured data of SIDER 110 and the HTML analysis of Daily Med 120 and Drugs.com 130 may be extracted 230, 235, and combined 240 to create an integrated drug list. These two lists are then combined using at least the drug-side effect data of SIDER to create 250 the Knowledge Base 140 of drug-side effect combinations.

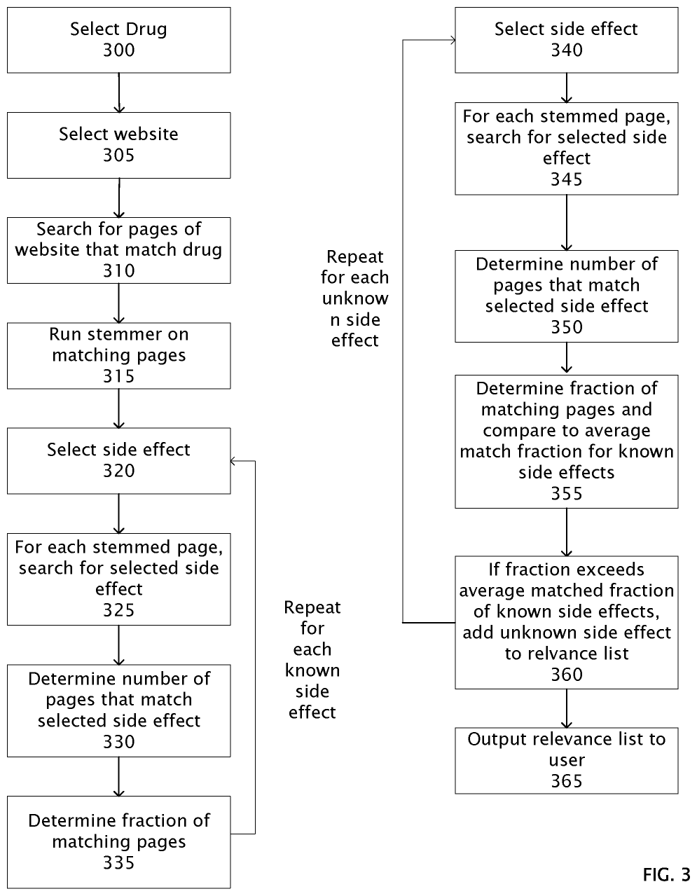

FIG. 3 is a block diagram of the method of identifying drug-side effect combinations from web discussions described in connection with the system of FIG. 1. The illustrated method may begin by selecting a drug 300 to analyze and selecting a website 305 to scan. The pages of the website may be scanned 310 to identify the pages that match the drug name of the selected drug. A stemmer algorithm may reduce 315 those pages to their stemmed form to facilitate matching, and then the pages matching each side effect 320 from the Knowledge Base 140 may be located 325 and counted 330. The counts may be used to calculate the average fraction of pages 335 that match a known side effect, which may be used as a relevance threshold, for example, using the Roccio method. Each side effect from the Knowledge Base 140, which is not already associated with the drug 340 (the unknown side effects), may then be scanned for 345, and the matching pages may be counted 350. For each unknown side effect, the fraction of matching pages may be determined 355, and the result may be compared with the fraction of matching pages for the known side effects determined in step 335. If the unknown fraction from step 355 is greater than (or, in some embodiments, about equal to) the known fraction 335, the unknown side effect may be added 360 to a list of relevant side effects. Once the scanning has been completed for all of the side effects, the list of relevant side effects may be output to a user 365. In some embodiments, rather than scanning all of the side effects, the user may select a side effect as well as a drug to scan for, or only a subset of the universe of side effects may be searched (e.g., side effects that are known to be associated with a related drug, or side effects related to a particular body system).

The procedure above may not discriminate between side effects and primary therapeutic effects of drugs. Thus, results may include not only that a drug may have a particular side effect, but also that it has its own therapeutic effect. For example, hypertensive medication may list “lowering blood pressure” as a “side” effect. This feature is not expected to be problematic, since a user may be able to distinguish side effects from therapeutic effects, but it may also be reduced or eliminated by using structured drug data from online sources as described above to identify therapeutic effects and temporarily remove them from the lexicon for analysis of that drug.

The above procedure rests on the assumption that all discussions about a drug and a side effect can be used to confirm their association. However, this assumption may not always hold since the discussions may convey negative meaning. For example, a user may mention that he or she does not have a side effect. If such cases happen frequently in the data set, the results of the method described above might not be valid, since a discussion about not having a side effect might be mistakenly considered as the one mentioning the side effect. In data sets where it is suspected or known that individuals may often discuss side effects that they do not have, industry-proven machine learning models for semantic analysis may be used to train the model with drug ingredients and drug names, so that the logical form returned from these models may be parsed to return positive (experienced discussed side effect) or negative (did not experience discussed side effect) review about a particular drug. In some such embodiments, to write the context-free grammar for the drugs, Backus-Naur form or the DCG (definite clause grammar) form may be used. The returned score range from 0-1 may then be used to validate the drug review as being a positive or a negative review, and only reviews exceeding some threshold as positive may be counted as “matching” the side effect. This threshold may be predetermined before applying the algorithm (e.g., 0.1, 0.2, 0.3, 0.4, 0.5, 0.6, 0.7, 0.8, or 0.9), or it may be dynamically determined, for example, using the Roccio method.

In embodiments that track vast amounts of data from an extremely large number of sources over a long period of time, the risk of data manipulation by third parties or patients whose behavior or experience are outliers is expected to be minimized. The data may be statistically analyzed to increase reliability with extremely large samples of data annotated. For example, human reviewers using Amazon Mechanical Turk may be used.

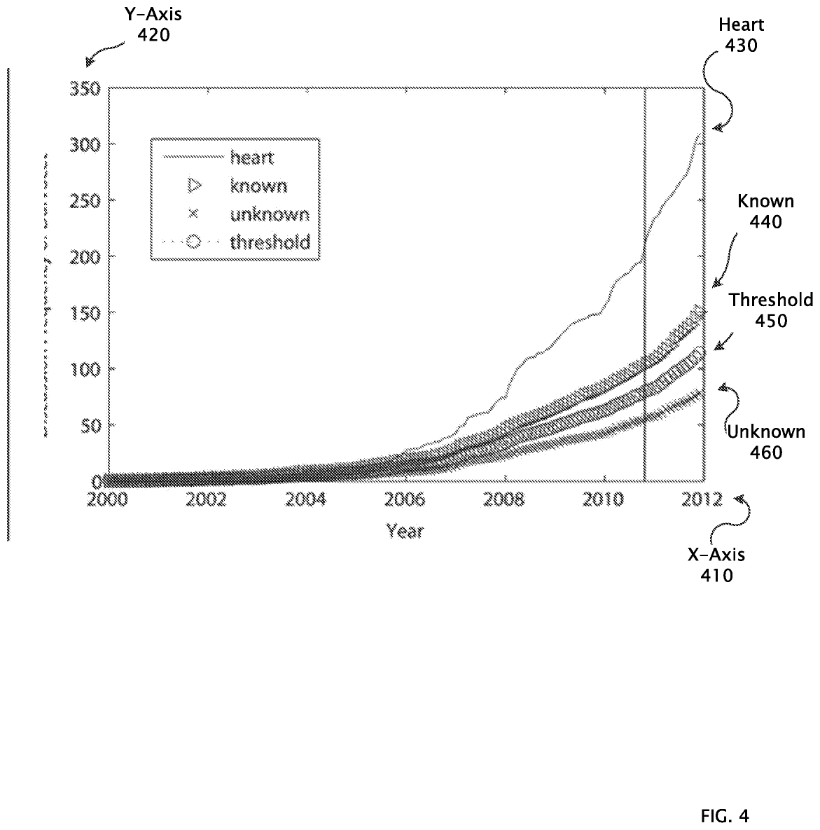

A proof of concept has shown that online discussions provide useful information discovering unrecognized drug side effects. FIG. 4 illustrates the results of the preliminary research for Darvocet as an example, which was recalled by the FDA on Nov. 19, 2010, for its risk of abnormal heart rhythms, which may cause sudden death. The x-axis 410 is the timeline, and the y-axis 420 is the cumulative discussion frequency. The lines labeled as Known 440 and Unknown 460 represent the average accumulated discussion frequencies for known and unknown drug side effects, respectively. Threshold 450 is in the middle of these two lines, indicating the classification boundary. At any given time, if the accumulated discussion frequency of the side effect is larger than the corresponding value at the classification boundary, the side effect will be predicted as relevant to the drug. Looking at empirical data about Heart 430, the solid line, it is clear that many discussions occurred about the side effect from at least about 2006, four years earlier than the official recall.

To quantitatively compare an implementation, another set of experiments may be conducted by leveraging FAERS, a database with drug side effect related reports that have been submitted to the FDA. FAERS contains the information about drug side effects gathered from a different channel than the one described above, and so can be leveraged to compare methods. FAERS maintains a record of side effect cases, which are utilized by the FDA to make the official recall/warning decisions. This information may be reported by physicians or patients, but the side effect is not confirmed until official announcements by drug companies or by the FDA. The evaluation measure used for this comparison may be precision and recall, which are basic measures used in information retrieval. In particular, precision measures the percentage of predicted drug side effects that are covered by FAERS. It may be computed by dividing the number of drug side effects that are both discovered by a method and reported in FAERS system with the number of drug side effects discovered by the method. Recall measures the percentage of drug side effects reported in FAERS that are also predicted by the method. It is computed by dividing the number of side effects that are both discovered by the method and reported in FAERS system with the number of side effects from the FAERS system.

Unlike drugs, not every side effect has a specific name, so it is possible that identifying all side effects by mining the text with string matching could miss some reported side effects. As a result, a “gold model” may be developed as a comparison for data that is validated for the top 200 drugs by a pharmacist using Amazon Mechanical Turk.

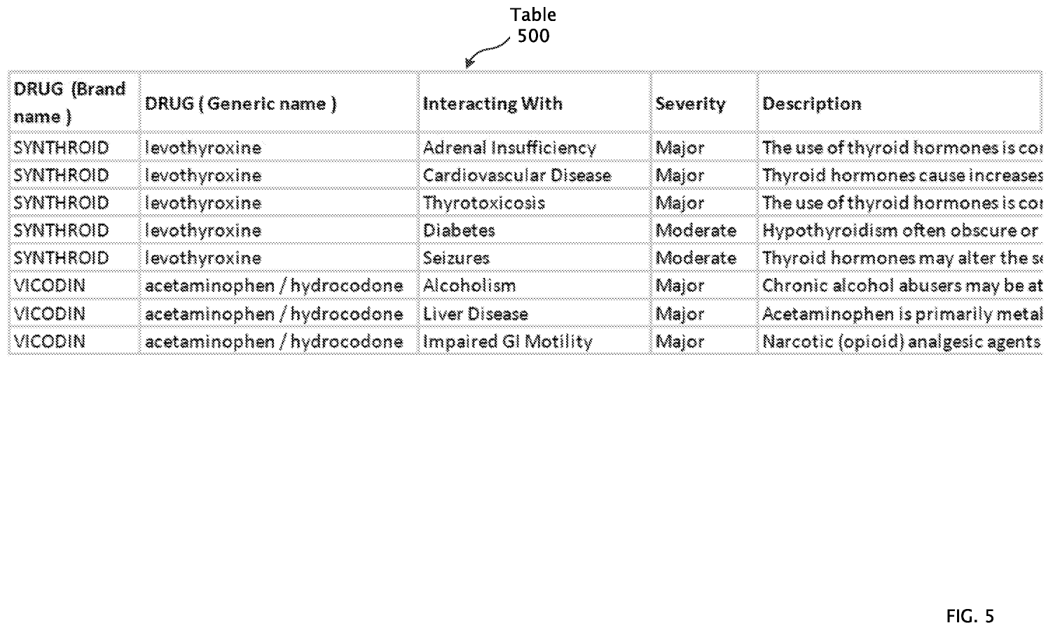

FIG. 5 shows a small portion of Table 500 containing drug-disease interaction data. For example, such a table may include a brand name of a drug, a generic name of the drug, an area of concern with which the drug may interact, a severity of the interaction, and a description.

While various aspects and embodiments have been disclosed herein, other aspects and embodiments will be apparent to those skilled in the art. The various aspects and embodiments disclosed herein are for purposes of illustration and are not intended to be limiting, with the true scope and spirit of the invention being indicated by the following claims.

The side effect database may be maintained by using continuous updates and periodic data ingestion. Analysis and predictions of additional previously unrecorded Drug-Drug-Interactions (DDI) may be performed with an industry-proven machine learning model for label propagation on the recorded interactions. The model may be trained with recorded DDIs and corresponding chemical substructures of a drug pair. The model may logically return potential DDIs based on the similarity between the patterns of each chemical substructure by clustering similar sample pairs of drugs toward a pair of drugs that have recorded interactions. A higher propagated chance may be predicted for sample pairs closer to the recorded pair. The returned propagated chance may range from 0 to 1, and the propagated chance may be sent to a pharmacist team for further testing verifying the authenticity of the chance of interaction before it is ingested into the database.

The side effect database may also include atomizing the database down to the ingredient level, where a drug would be the combination of multiple ingredients, and each ingredient may have their own side effect and interactions accordingly. The atomization may allow the machine learning model to propagate the label of the interactions between ingredients, exploring the possibility to predict multiple interactions between a single pair of drugs based on different ingredient combinations.

1. A method for determining whether a search drug has a side effect, comprising:
searching a target website to identify pages matching the search drug;
searching the identified pages for text matching the side effect; and
determining relevance of the side effect by comparing a fraction of identified pages matching the side effect to a threshold, wherein a fraction of identified pages greater than or about equal to the threshold indicates that the side effect is relevant to the search drug.

2. The method of claim 1, further comprising accessing a database of drugs to select the search drug.

3. The method of claim 1, further comprising accessing a database of side effects to select the side effect.

4. The method of claim 1, wherein the search drug is an active ingredient.

5. The method of claim 1, wherein the search drug is an inactive ingredient.

6. The method of claim 1, wherein the target website includes health-related user-generated content.

7. The method of claim 6, wherein the target website is a health-related forum.

8. The method of claim 6, wherein the target website is a social community.

9. The method of claim 1, wherein identifying pages matching the search drug includes identifying a drug name field in a structured page of the target website.

10. The method of claim 1, wherein identifying pages matching the search drug includes matching a name of the search drug to text on the target website.

11. The method of claim 1, wherein searching the identified pages for text matching the side effect includes preprocessing the identified pages to normalize text.

12. The method of claim 11, wherein preprocessing the identified pages includes applying a Porter Stemmer algorithm.

13. The method of claim 1, wherein searching the identified pages for text matching the side effect includes identifying text strings having elements that overlap elements of the side effect.

14. The method of claim 1, wherein searching the identified pages for text matching the side effect includes using semantic analysis to determine whether the text indicates that the side effect did not occur.

15. The method of claim 14, wherein text determined to indicate that the side effect did not occur is determined to not match the side effect.

16. The method of claim 1, wherein the threshold is determined using a Rocchio method.

17. The method of claim 1, further comprising searching the target website to identify pages matching a second search drug.

18. The method of claim 17, wherein identifying pages matching the search drug includes identifying pages matching both the search drug and the second search drug.

19. A system for determining whether a search drug may have a side effect, comprising:
a first search engine that searches a target website to identify pages matching the search drug;
a second search engine that searches the identified pages for text matching the side effect; and
a relevance calculator that determines relevance of the search side effect by comparing a fraction of identified pages matching the side effect to a threshold, wherein a fraction of identified pages greater than or about equal to the threshold indicates that the side effect is relevant to the search drug.

20. A method for constructing a side effect database for a group of drugs, comprising:
obtaining a side effect lexicon including a listing of possible side effects;
creating a drug database including a record for each drug of the group of drugs; and
for each drug of the group of drugs,
identifying a plurality of web pages that include a discussion of the drug;
for each pair of (i) web page of the identified plurality and (ii) side effect of the listing,
locating any text strings in the web page that match the side effect;
calculating a relevance of each side effect to the drug by considering located matches for all web pages that include a discussion of the drug; and
if the calculated relevance exceeds a threshold, storing an indicator of the calculated relevance of the side effect to the drug in the database.
